(19) United States
(12) Patent Application Publication
(10) Pub. No.: US 2011/0136772 A1
(43) Pub. Date: Jun. 9, 2011

(54) SUGAR COATINGS AND METHODS THEREFOR

(75) Inventors: John C. Clark, Peru, NY (US); John J. Michelucci, Plattsburgh, NY (US); Deborah M. Sherman, Plattsburgh, NY (US)

(73) Assignee: Wyeth LLC, Madison, NJ (US)

(21) Appl. No.: 13/017,770

(22) Filed: Jan. 31, 2011

Related U.S. Application Data
(63) Continuation of application No. 11/146,732, filed on Jun. 7, 2005, now abandoned.
(60) Provisional application No. 60/577,668, filed on Jun. 7, 2004.

(51) Int. Cl. A61K 31/56; A61P 15/00

(52) U.S. Cl. 514/178

(57) ABSTRACT

Compositions particularly useful as coatings for solid dosage forms of therapeutic agents are provided, as are solid dosage forms comprising such coatings, processes for preparing such solid dosage forms, and the products of those processes. The coating compositions generally provide excellent strength and resistance to cracking, even when applied to flexible/swellable tablet cores such as hydrogel-type cores. The compositions also exhibit excellent odor-blocking characteristics.

### CROSS-REFERENCE TO RELATED APPLICATION

This application claims priority to U.S. Provisional Application Ser. No. 60/577,668, filed on Jun. 7, 2004, which is incorporated herein by reference in its entirety.

### FIELD OF THE INVENTION

The invention is directed generally to the field of pharmaceutical formulations. More specifically, the invention relates to sugar-containing compositions suitable for use in coating solid preparations such as tablets, pills, granules and grains. Methods of using such coatings are provided, as are solid dosage forms coated with the compositions.

### BACKGROUND OF THE INVENTION

Solid pharmaceutical dosage forms, most notably tablets, have been coated using a wide variety of materials and processes. The reasons for this include the aesthetic as well as the practical. For example, tablet coatings can mask an unpleasant taste or odor, can increase ease of ingestion by the patient and can serve to improve the ultimate appearance of the dosage form. Similarly, coatings can protect a product from the effects of air, moisture and light, can improve product identification and can facilitate handling in packaging and filling lines during manufacture.

Typically, pharmaceutical dosage forms can be sugar coated or film coated. Conventional sugar coating compositions have been prepared as aqueous solutions of sugar (syrups), most commonly sucrose, and it is said that tablets with excellent hermetic properties and smooth surface are obtained by the formation of tight, block-form structures of sugar crystals. Additional agents that may be included in sugar-coated tablets include talc (used as a sugar coating dispersion agent), precipitated calcium carbonate (used as a sugar coating suspension agent), gelatin, gum arabic and pullulan (used as sugar coating binders), and carnauba wax (often applied as a sugar coating glossing agent).

Unfortunately, sugar-coating is a multi-step and tedious process, and is highly dependent on the use of skilled manpower. The typical sugar-coating process can be subdivided into six main steps: (1) sealing; (2) subcoating; (3) smoothing; (4) color coating; (5) polishing; and (6) printing. In the hands of a skilled worker, sugar coated products are elegant in appearance, but certain problems beset the process and the ultimate product. For example, the sugar coating process requires that the tablets be kept constantly tumbling, thus presenting difficulties such as fragmenting of those units not strong enough to withstand the stress encountered. Also, color nonuniformity, rough or overly soft coatings and/or marbling may present additional problems to be addressed.

In an attempt to overcome some of these difficulties, film coating was introduced to the pharmaceutical industry in the 1950's. Film coating involves the deposition of a thin, uniform, typically polymeric membrane to the substrate, usually by a spray technique. Advantages of the film coating process include minimal weight increase of the ultimate dosage form, reduction in processing times, and improved resistance to chipping.

Film coatings also have been utilized to modify the release of the constituents of the core tablet as, for example, via enteric coatings in which the polymer employed in the coating is essentially impervious to gastric pH but is soluble in the increased pH of the intestines. A further application of film coating lies in the formulation of extended-release coatings that help eliminate the need for multiple dose regimens of a particular therapeutic agent. Various types of extended release approaches are known. One such approach is a diffusion coating, which involves depositing a coating (usually from an organic solvent) on a soluble substrate core with a porous membrane that is water-permeable but water-insoluble. The release profile of the therapeutic agent can be modified by the inclusion of water-soluble substances within the membrane. These substances are dissolved by the gastrointestinal fluids, thereby creating pores within the film. These pores allow the gastrointestinal fluids to pass through the membrane and dissolve the therapeutic agent within the tablet core. The diffusion rate can be controlled by the thickness and composition of the diffusion membrane. For this system to function properly, the constituents of the diffusion coating formulation should exhibit good mechanical strength and flexibility. Unfortunately, however, such coating formulations, when deposited on the substrate, frequently lack the requisite mechanical strength and flexibility, thereby leading to rupture of the deposited film during dissolution in the gastrointestinal tract. This, in turn, permits the sudden release of the entire contents of the substrate containing the therapeutic agent in a phenomenon referred to as “dose dumping”. Such a situation is clearly undesirable for extended-release dosage forms given the higher amount of therapeutic agent found therein as compared to conventional formulations.

Additionally, notwithstanding the advantages that film-coating provides, certain difficulties attend the film coating process, including the tendency to laminate if the tablets being coated are not of sufficient strength, the inability to hide defects in the tablet core, mottling and the like. Ironically, the use of organic solvents in film coating, which permits a number of process advantages, also presents some of the major disadvantages. Due to their volatility, the use of organic solvents in the film coating process can lead to flammability hazards as well as concerns over environmental effects and potential toxicity to the operators. Organic solvents also add to the cost of the overall process, due to the costs of the solvents per se or costs encountered in reducing any potential hazards thereof.

Moreover, film-coating may not be suitable for tablets that contain a particularly hygroscopic core that is apt to swell either during processing or storage. For example, film-coated tablets with hydrogel cores, which contain relatively high percentages of water-soluble cellulosic materials in the tablet cores, have a tendency to crack. Tablets with cracked coatings are unacceptable, from both an aesthetic and functional standpoint; the elegant appearance, ease of ingestion, and odor-masking properties are diminished, and the active ingredient in the tablet cores may become exposed to environmental conditions detrimental to product stability.

The present invention addresses one or more of these problems by providing coatings that exhibit good mechanical strength and flexibility when applied to a substrate, thereby reducing the possibility of cracking. The coating formulation of the present invention also utilizes an aqueous solvent system thereby eliminating or at least minimizing the use of organic solvents in the film coating process delineated above. At the same time, the compositions generally can be applied using traditional film-coating equipment, and thus may be utilized in simpler and less labor-intensive methods than are necessary to produce the sugar-coated tablets of the prior art.

### SUMMARY OF THE INVENTION

In one embodiment, the invention is directed to compositions comprising water and a solids component that comprises: from about 30 weight percent (%) to about 60 weight % of at least one sugar; from about 5 weight % to about 10 weight % of at least one binder; from about 3 weight % to about 10 weight % of at least one hydroxyalkyl cellulose; from about 15 weight % to about 50 weight % of at least one water soluble polymer; optionally, a therapeutic agent in an amount of up to about 3 weight %; optionally, a second polymer that is water soluble or water dispersible, in an amount of up to about 20 weight %; and optionally, at least one plasticizer in an amount of up to about 8 weight %.

The invention also is directed to solid dosage forms comprising a core material and one or more coatings disposed thereon, wherein the coating comprises: from about 30 weight % to about 60 weight % of at least one sugar; from about 5 weight % to about 10 weight % of at least one binder; from about 3 weight % to about 10 weight % of at least one hydroxyalkyl cellulose; from about 15 weight % to about 50 weight % of at least one water soluble polymer; optionally, a therapeutic agent in an amount of up to about 3 weight %; optionally, a second polymer that is water soluble or water dispersible, in an amount of up to about 20 weight %; and optionally, at least one plasticizer in an amount of up to about 8 weight %.

In some further embodiments, the coating can include one or more therapeutic agents. In some such embodiments, the coating further includes one or more polymers that provide control of the release of the therapeutic agent from the coating.

In another embodiment, the invention is directed to processes comprising: providing a tablet core; spraying or otherwise applying to said tablet core a sugar coating composition comprising water and a solids component that comprises: from about 30 weight % to about 60 weight % of at least one sugar; from about 5 weight % to about 10 weight % of at least one binder; from about 3 weight % to about 10 weight % of at least one hydroxyalkyl cellulose; from about 15 weight % to about 50 weight % of at least one water soluble polymer; optionally, a therapeutic agent in an amount of up to about 3 weight %; optionally, a second polymer that is water soluble or water dispersible, in an amount of up to about 20 weight %; and optionally, at least one plasticizer in an amount of up to about 8 weight %; resulting in a coated tablet core.

In further embodiments, the invention is directed to the products of the foregoing processes.

### DESCRIPTION OF THE DRAWINGS

FIG. 1 shows plasma MPA levels in six beagle dogs following oral administration of the tablet formulation described in Example 3.

### DETAILED DESCRIPTION OF THE INVENTION

Concentrations, amounts, percentages, and other numerical data may be expressed or presented herein in a range format. It is to be understood that such a range format is used merely for convenience and brevity and thus, should be interpreted flexibly to include not only the numerical values explicitly recited as the limits of the range, but also, to include each of the individual numerical values or sub-ranges encompassed within that range as if each numerical value and sub-range is explicitly recited.

As an illustration, a concentration range of “about 1 weight % to about 10 weight %” should be interpreted to include not only the explicitly recited concentration of about 1 weight % to about 10 weight %, but also individual concentrations and the sub-ranges within the indicated range. Thus, included in this numerical range are individual concentrations such as 2 weight %, 5 weight %, and 8 weight %, and sub-ranges such as from 1 weight % to 3 weight %, from 5 weight % to 9 weight %, etc. The same principle applies to ranges reciting only one numerical value.

Similarly, an open ended range recited as “less than about 10 weight %” should be interpreted to include all of the values and ranges as elaborated above. Furthermore, it is understood that functional limitations may exist for limits not expressly recited by an open ended range, and that such limitations are included inherently as part of the disclosure of the present application, though not expressly recited. Such an interpretation should apply regardless of the breadth of the range or the characteristics being described.

In one embodiment, the invention is directed to compositions suitable for use in coating a solid preparation, such as a tablet, pill, granule, or the like. The compositions comprise water, and a solids component, and also optionally may include a therapeutic agent. Up to about 50% by weight of water may be replaced by another solvent, such as an alcohol. Preferably, the composition is in the form of an aqueous suspension, obtained by combining from about 30 weight % to about 98 weight % water, and from about 2 weight % to about 70 weight % of the solids component. In certain embodiments, the composition comprises from about 75 weight % to about 85 weight % water and from about 15 weight % to about 25 weight % of the solids component. In one such embodiment, the composition comprises about 82 weight % water.

The solids component contains, inter alia, one or more sugars. As used herein, the term “sugar” refers to any type of simple carbohydrate, such as a mono or disaccharide, either naturally obtained, refined from a natural source, or artificially produced, and includes, without limitation, sucrose, dextrose, maltose, glucose, fructose, galactose, mannose, lactose, trehalose, lactulose, levulose, raffinose, ribose, and xylose. The term “sugar,” as used herein, also includes various “sugar substitutes” widely known to those of ordinary skill in the art of preparing solid dosage forms, such as the polyhydric alcohols (sometimes referred to as “sugar alcohols” or hydrogenated saccharides), for example sorbitol, mannitol, xylitol, and erythritol, and the sugar derivatives of polyhydric alcohols, such as maltitol, lactitol, isomalt, and polyalditol. Accordingly, the recitation of the term “sugar” generically should be interpreted to include such specific compounds, as well as others not expressly recited. In certain embodiments, the solids component of compositions of the invention include at least one sugar that is a mono- or disaccharide, for example, sucrose, dextrose, maltose, glucose, fructose, galactose, mannose, lactose. In some such embodiment, the sugar is sucrose.

Unlike traditional sugar coatings, which are produced using syrups that contain upwards of 80 weight % sugars, on a dry weight basis, the compositions of the present invention contain a solids component that comprises from about 30 weight % to about 60 weight % of at least one sugar, for example, sucrose. In some embodiments, the solids component contains from about 35 weight % to about 55 weight % sugar. In still other embodiments, the solids component contains from about 35 to about 45 weight % sugar.

The solids component also contains a binder, such as microcrystalline cellulose or powdered cellulose, in an amount of from about 5 weight % to about 10 weight %. In some embodiments, the binder is present in an amount of from about 5.5 weight % to about 9 weight %, or in further embodiments, in an amount of from about 5.5 to about 7.5 weight %.

The solids component further contains from about 3 weight % to about 10 weight % of at least one hydroxyalkyl cellulose in which the alkyl group has from between one and ten carbon atoms. Representative hydroxyalkyl celluloses, without limitation, include hydroxyethyl cellulose (HEC) and hydroxypropyl cellulose (HPC). In some embodiments, the hydroxyalkyl cellulose is HPC. In some embodiments, the solids component comprises from about 4 weight % to about 7 weight % hydroxyalkyl celluloses; and in further embodiments, from about 4 to about 5.5 weight % hydroxyalkyl celluloses.

In addition, the solids component preferably contains from about 15 weight % to about 50 weight % of at least one water soluble polymer. Water soluble polymers suitable for use in coatings and as excipients in pharmaceutical dosage forms are widely known to those of skill in the art, and include, inter alia, Hypromellose (hydroxypropyl methylcellulose), acacia, sodium carboxymethylcellulose, dextrin, alginic acid, gelatin, guar gum, methylcellulose, sodium alginate, zein, polyvinylpyrrolidone, vinylpyrrolidine-vinyl acetate copolymer, vinyl acetate-crotonic acid copolymer and ethyl acrylate-methacrylate acid copolymer. In some embodiments, the water soluble polymer is Hypromellose. In other embodiments, the polymer is sodium carboxymethylcellulose; and still other embodiments, methylcellulose. The specific amount of water soluble polymer to be used in the solids component varies, depending upon the polymer selected. In some embodiments, the water soluble polymer is present in an amount from about 25 weight % to about 40 weight %, and further, in an amount from about 20 weight % to about 30 weight %.

The solids component of the compositions also contains at least one plasticizer. Suitable plasticizers also are well known to those skilled in the art, and include, for example, propylene glycol, glycerin, trimethylolpropane, polyethylene glycol (PEG) polymers, dibutyl sebacate, acetylated monoglycerides, diethylphthalate, triacetin, glyceryltriacetate, acetyltrietyhyl citrate and triethyl citrate. In certain embodiments, a PEG polymer is used. Such polymers are available commercially by grades of average molecular weight such as PEG 100 to PEG 4,000. In some such embodiments, PEG 400 is used. The plasticizer usually is present in the solid components in an amount of from about 0 weight % to about 8 weight %. In some embodiments, the plasticizer is present in an amount from about 5 weight % to about 7 weight %, or from about 2 weight % to about 4 weight %, or from about 2.5 to about 3.5 weight %.

The solids component of the compositions also may contain a second polymer, which is water soluble, or which is water dispersible, i.e., capable of forming an aqueous dispersion. Suitable polymers that can be used as, for example, a second polymer, without limitation, are polyvinyl acetates such as Kollicoat® SR30D (BASF Corp., Mt. Olive, N.J.), ethylcelluloses such as Aquacoat® ECD (FMC Corp., Philadelphia, Pa.), polymethacrylates such as Eudragit® NE30D, NE40D, RS30D and RL30D (Degussa Corp., Parsippany, N.J.) and Kollicoat® MAE30DP (BASF Corp.) and polyvinyl alcohols such as Opadry® AMB (Colorcon, West Point, Pa.). In some embodiments, the second polymer may be a mixture of one or more suitable polymers, for example, a mixture of RS30D and RL30D; and thus, the solids component of the compositions would comprise at least two additional polymers. In some embodiments, the second polymer is a polymethacrylate. In some such embodiments, the polymethacrylate contains neutral methyacrylic acid esters. In some such embodiments, the polymethacrylate contains neutral methyacrylic acid esters having trimethylammonioethyl methacrylate chloride in a molar ratio of quaternary ammonium groups to neutral ester groups of 1:20; or in further embodiments, of 1:40. In some embodiments, the polymethacrylate contains neutral methyacrylic acid esters without any functional groups. As used herein, the term “functional groups” refers to atoms or small groups of atoms (for example, two to four atoms) that exhibit a characteristic reactivity when treated with certain reagents; for example, and without limitation, the term includes, alkanes, alkenes, alkynes, alkyl halides, benzene, amines, ammonium, ethers, alcohols, aldehydes, ketones and carboxylic acids. In some embodiments, the second polymer that is water soluble or water dispersible is in an amount from about 3 weight % to about 20 weight % based on the percentage of solids in the solids component of the composition.

The compositions also may include optional ingredients, such as other flavoring agents, artificial sweeteners, color pigments, lubricants, glidants, surfactants. The selection of such alternative ingredients is within the skill of those in the art.

As noted previously, the compositions also may include one or more therapeutic agents. As used herein, the term “therapeutic agent” also refers to a substance which is capable of exerting a therapeutic biological effect in vivo. The therapeutic agents may be neutral or positively or negatively charged. Examples of suitable pharmaceutical agents include, inter alia, diagnostic agents, pharmaceuticals, drugs, synthetic organic molecules, proteins, peptides, vitamins, and steroids. For example, the composition may include one or more hormonal steroids, such as medroxyprogesterone acetate, levonorgestrel, gestodene, medrogestone, estradiol, estriol, ethinylestradiol, mestranol, estrone, dienestrol, hexestrol, diethylstilbestrol, progesterone, desogestrel, norgestimate, hydroxyprogesterone, norethindrone, norethindone acetate, norgestrel, megestrol acetate, methyltestosterone, ethylestrenol, methandienone, oxandrolone, trimegestone, dionogest, and the like. Additionally, tissue selective progesterones and/or progesterone antagonists, which may or may not have the typical steroidal functionality, may be present in the composition. These include, but are not limited to: RU-486 (mifepristone), ZK 98 299 (onapristone), ZK-137316 (Schering AG, Berlin), ZK-230211 (Schering AG, Berlin), and HRP-2000 (17-acetoxy-[11β-(4-N,N-dimethylaminophenyl)]-19-norpregna-4,9-diene-3,20-dione). Where desired, estrogenic steroids and progestogenic steroids may be used in combination.

The composition of the present invention is particularly suitable for use in coating a core material to produce a solid dosage form. The term “core material” refers to any tablet, caplet, particle, micronized particle, particulate, pellet, pill, core, powder, granule, granulate, small mass, seed, specks, spheres, crystals, beads, agglomerates, mixtures thereof and the like. Typically, the preferred core material will be in a form sufficiently stable physically and chemically to be effectively coated in a system that involves some movement of the tablet, as for example, in a perforated coating pan.

In a preferred embodiment, the core material is present in the form of a tablet. As used herein, the term “tablet” refers to a solid pharmaceutical dosage form containing a therapeutic agent with or without suitable diluents and prepared by either compression or molding methods, such as are well known to those of ordinary skill in the art. Suitable methods of forming tablets are described, for example, in Edward M Rudnick, et al., “Oral Solid Dosage Forms,” in Remington: The Science and Practice of Pharmacy, 20^{th} Ed., Chap. 45, Alfonso R. Gennaro, ed., Philadelphia College of Pharmacy and Science, Philadelphia, Pa. (2000), herein incorporated by reference in its entirety. In more preferred embodiments, the core material is a tablet formed by compression methods.

Most frequently, the core material will comprise at least one therapeutic agent, as defined previously, and at least one pharmaceutically acceptable excipient. The term “pharmaceutically acceptable,” as used herein, refers to materials that are generally not toxic or injurious to a patient when used in the compositions of the present invention, including when the compositions are administered by the oral route. The term “patient,” as used herein, refers to animals, including mammals, preferably humans. “Excipients,” as that term is used herein, refers to ingredients that provide bulk, impart satisfactory processing and compression characteristics, help control the dissolution rate, and/or otherwise give additional desirable physical characteristics to the core material. Included within this term, for example, are diluents, binders, lubricants and disintegrants well known to those of ordinary skill in the art, as described, for example, in the Handbook of Pharmaceutical Excipients, American Pharmaceutical Association, Washington, D.C. and The Pharmaceutical Society of Great Britain, London, England (1986), herein incorporated by reference in its entirety. Suitable excipients may include, for example, cellulosic material, such as, Hypromellose, HPC, HEC, carboxymethylcellulose, microcrystalline cellulose, ethyl cellulose, methyl cellulose, and their derivatives and salts; other organic compounds, such as PEG, talc, lactose and other sugars (as described above), acacia, dextrin, alginic acid, ethylcellulose resin, gelatin, guar gum, methylcellulose, pregelatinized starch, sodium alginate, starch, zein, polyvinylpyrrolidone, vinylpyrrolidine-vinyl acetate copolymer, vinyl acetate-crotonic acid copolymer and ethyl acrylate-methacrylate acid copolymer; plasticizers, such as propylene glycol, glycerin, trimethylolpropane, PEG polymers, dibutyl sebacate, acetylated monoglycerides, diethylphthalate, triacetin, glyceryltriacetate, acetyltrietyhyl citrate and triethyl citrate; and lubricants, such as talc, magnesium stearate, calcium stearate, stearic acid, hydrogenated vegetable oils, magnesium lauryl sulfate, sodium benzoate, a mixture of sodium benzoate and sodium acetate, sodium chloride, leucine, and Carbowax® 4000.

A wide variety of therapeutic agents may be utilized in the core material. Specific examples of therapeutic agents include, but are not limited to: acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenytoin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenytoin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, vitamin K-S (II) and zafirlukast. Additional therapeutic agents include abacavir, acebutolol, acrivastine, alatrofloxacin, albuterol, albendazole, alfentanil, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, apomorphine, astemizole, atenolol, atropine, azathioprine, azelastine, azithromycin, baclofen, benethamine, benidipine, benzhexol, benznidazole, benztropine, biperiden, bisacodyl, bisanthtene, bromazepam, bromocriptine, bromperidol, brompheniramine, brotizolam, bupropion, butenafine, butoconazole, cambendazole, cam ptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclo-cycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyl-imidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, modafinil, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfioxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxatidine, roxithromycin, salbutamol, saquinavir, selegiline, sertraline, sibutramine, sildenafil, sparfloxacin, spiramycins, stavudine, sufentanil, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₅, vitamin K₆, vitamin K₇, zafirlukast, zolmitriptan, zolpidem and zopiclone. Of course, any of the foregoing therapeutic agents may be included in the coating composition, as discussed previously, and any of the therapeutic agents discussed with regard to the coating composition alternatively may be included in the core material.

The core material may be designed for delivering therapeutic agents intended to be delivered over a sustained period of time. The following are representative of such therapeutic agents: anti-inflammatory, antipyretic, anti-spasmodics or analgesics such as indomethacin, diclofenac, diclofenac sodium, codeine, ibuprofen, phenylbutazone, oxyphenbutazone, mepirizole, aspirin, ethenzamide, acetaminophen, aminopyrine, phenacetin, butylscopolamine bromide, morphine, etomidoline, pentazocine, fenoprofen calcium, naproxen, selecxip, valdecxip, and tolamadol, anti-rheumatism drugs such as etodolac, anti-tuberculoses drugs such as isoniazide and ethambutol hydrochloride, cardiovascular drugs such as isosorbide dinitrate, nitroglycerin, nifedipine, barnidipine hydrochloride, nicardipine hydrochloride, dipyridamole, amrinone, indenolol hydrochloride, hydralazine hydrochloride, methyldopa, furosemide, spironolactone, guanethidine nitrate, reserpine, amosulalol hydrochloride, lisinopril, metoprolol, pilocarpine, and talcetin, antipsychotic drugs such as chlorpromazine hydrochloride, amitriptyline hydrochloride, nemonapride, haloperidol, moperone hydrochloride, perphenazine, diazepam, lorazepam, chlorodiazepoxide, adinazolam, alprazolam, methylphenidate, myrnasipran, peroxetin, risperidone, and sodium valproate, anti-emetics such as metoclopramide, lamocetron hydrochloride, granisetron hydrochloride, ondansetron hydrochloride, and azacetron hydrochloride, antihistamines such as chlorpheniramine maleate and diphenhydramine hydrochloride, vitamins such as thiamine nitrate, tocopherol acetate, cycothiamine, pyridoxal phosphate, cobarnamide, ascortic acid, and nicotinamide, anti-gout drugs such as allopurinol, colchicine, and probenecide, anti-Parkinson's disease drugs such as levodopa and selegrine, sedatives and hypnotics such as amobarbital, bromuralyl urea, midazolam, and chloral hydrate, antineoplastics such as fluorouracil, carmofur, acralvidine hydrochloride, cyclophosphamide, and thiodepa, anti-allergy drugs such as pseudoephedrine and terfenadine, decongestants such as phenylpropanolamine and ephedorine, diabetes mellitus drugs such as acetohexamide, insulin, tolbutamide, desmopressin, and glipizide, diuretics such as hydrochlorothiazide, polythiazide, and triamterene, bronchodilators such as aminophylline, formoterol fumarate, and theophylline, antitussives such as codeine phosphate, noscapine, dimorfan phosphate, and dextromethorphan, anti-arrhythmics such as quinidine nitrate, digitoxin, propafenone hydrochloride, and procainamide, topical anesthetics such as ethyl aminobenzoate, lidocaine, and dibucaine hydrochloride, anti-convulsants such as phenytoin, ethosuximide, and primidone, synthetic glucocorticoids such as hydrocortisone, prednisolone, triamcinolone, and betamethasone, antiulceratives such as famotidine, ranitidine hydrochloride, cimetidine, sucralfate, sulpiride, teprenone, plaunotol, 5-aminosalicylic acid, sulfasalazine, omeprazole, and lansoprazol, central nervous system drugs such as indeloxazine, idebenone, thiapride hydrochloride, bifemelane hydrocide, and calcium homopantothenate, antihyperlipoproteinemics such as pravastatin sodium, simvastatin, lovastatin, and atorvastatin, antibiotics such as ampicillin hydrochloride, phthalylsulfacetamide, cefotetan, and josamycin, BPH therapeutic agents such as tamsulosin hydrochloride, doxazosin mesylate, and terazosin hydrochloride, drugs affecting uterine motility such as branylcast, zafylcast, albuterol, ambroxol, budesonide, and reproterol, peripheral circulation improvers of prostaglandin I derivatives such as beraprost sodium, anticoagulants, hypotensives, agents for treatment of cardiac insufficiency, agents used to treat the various complications of diabetes, peptic ulcer therapeutic agents, skin ulcer therapeutic agents, agents used to treat hyperlipemia, tocolytics, etc. The therapeutic agent can be used in its free form or as a pharmaceutically acceptable salt. Moreover, one or a combination of two or more therapeutic agents may be present in the core material.

In some embodiments, the therapeutic agent in the core material includes conjugated estrogens. “Conjugated estrogens” (CE) as used herein includes both natural and synthetic conjugated estrogens, such as the compounds described in the United States Pharmacopeia (USP 23), as well as other estrogens so considered by those skilled in the art. Further, “conjugated estrogens” refers to esters of such compounds, such as the sulfate esters, salts of such compounds, such as sodium salts, and esters of the salts of such compounds, such as sodium salts of a sulfate ester, as well as other derivatives known in the art. Some specific examples include: 17-alpha and beta-dihydroequilin, equilenin, 17-alpha and beta-dihydroequilenin, estrone, 17-beta-estradiol, and their sodium sulfate esters.

Although CE are typically a mixture of estrogenic components, such as estrone and equilin, the core material may be formulated to either utilize such a mixture, or to include only selected or individual estrogenic components. These CE may be of synthetic or natural origin. Examples of synthetically produced estrogens include, inter alia, sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate, estropipate and ethinyl estradiol. The alkali metal salts of 8,9-dehydroestrone and the alkali metal salts of 8,9-dehydroestrone sulfate ester, as described in U.S. Pat. No. 5,210,081, which is herein incorporated by reference, also may be used. Naturally occurring CE are usually obtained from pregnant mare urine and then are processed and may be stabilized. Examples of such processes are set forth in U.S. Pat. Nos. 2,565,115 and 2,720,483, each of which are herein incorporated by reference.

Many CE products are commercially available. Preferred among these is the naturally occurring CE product known as Premarin® (Wyeth, Madison, N.J.). Another commercially available CE product prepared from synthetic estrogens is Cenestin® (Duramed Pharmaceuticals, Inc., Cincinnati, Ohio). The specific CE dose included in the core material may be any dosage required to achieve a specific therapeutic effect, and may vary depending on the specific treatment indicated, and on the specific CE included in the tablet. However, in general, dosages of CE included in the tablet can range from about 0.1 mg CE/dosage form to about 5.0 mg CE/dosage form, with dosages of from about 0.3 mg CE/dosage form to about 2 mg CE/dosage form preferred. In some embodiments, the dosage of CE is from about 0.3 mg CE/dosage form, about 0.45 mg CE/dosage form, about 0.625 mg CE/dosage form, about 0.9 mg CE/dosage form, or about 1.25 mg CE/dosage form. Viewed alternatively, based on the total weight of the solid dosage form, on a dry weight basis, the amount of CE/dosage form may range from about 0.05 weight % to about 1.0 weight %, with amounts of from 0.1 weight % to about 0.3 weight % preferred.

Thus, in addition to the coating compositions described previously, the present invention also is directed to solid dosage forms comprising a core material and one or more coating disposed thereon, as each element has been described heretofore. In certain embodiments, the dosage form is a coated tablet. In some embodiments, the solid dosage forms comprise from about 30 weight % to about 70 weight % of the core material, and from about 30 weight % to about 70 weight % of the coating, and in still further embodiments, from about 40 weight % to about 60 weight % of the core material and from about 40 weight % to about 60 weight % of the coating. The solid dosage forms also can optionally include one or more additional coats. For example, a further sugar coating as described herein, disposed either on top of the coating previously described, or in between the core and coating. The solid dosage form also can include a color coat and/or a polish coat. In some embodiments, the color coat constitutes from about 0.5 weight % to about 10 weight % of the dosage form, and/or a polish coat that constitutes from about 0.1 weight % to about 5 weight % of the dosage form.

In some embodiments, the solid dosage form is in the form of a coated tablet, wherein the core material comprises at least one therapeutic agent and at least one pharmaceutically acceptable excipient. In some such embodiments, the excipient in the core material can include one or more cellulosic materials in a cumulative amount of from about 15 weight % to about 50 weight % of the solid dosage form; from about 18 weight % to about 40 weight % of the solid dosage form; or from about 40 weight % to about 45 weight % of the solid dosage form. In some such embodiments, the cellulosic material includes Hypromellose, microcrystalline cellulose, and sodium carboxymethylcellulose, and mixtures thereof.

In addition to the cellulosic material, in certain embodiments, the core material also includes a sugar excipient, such as lactose. A preferred form of lactose is lactose monohydrate, which is well known to those of skill in the art. In some embodiments, the dosage form of the invention is a coated tablet that comprises a tablet core containing from about 4 weight % to about 35 weight % lactose monohydrate, and from about 20 weight % to about 40 weight % cellulosic material based on the total weight of the solid dosage form. In a further such embodiment, the cellulosic material is a mixture of microcrystalline cellulose and Hypromellose. A lubricant, such as, for example, magnesium stearate, also can be utilized in the tablet core of such embodiments, typically, at an amount of up to about 1 weight % based on the total weight of the solid dosage form.

The formulations of the invention can be utilized with any of the aforementioned therapeutic agents, but is particularly suited for coated tablets that utilize conjugated estrogens, such as the conjugated estrogens desiccation with lactose. In some embodiments, from about 3 weight % to about 15 weight % of the conjugated estrogens desiccation with lactose (about 4.3 weight % conjugated estrogens), based on the total weight of the solid dosage form, is included in the tablet core. In still further such embodiments, about 4 weight % to about 10 weight % of the conjugated estrogens desiccation with lactose (about 4.3 weight % conjugated estrogens), is included in the tablet core. In certain embodiments, the conjugated estrogens are present in an amount from about 0.1 mg CE/dosage form to about 5.0 mg CE/dosage form, or from about 0.3 mg CE/dosage form to about 2 mg CE/dosage form. In some embodiments, the dosage of CE is from about 0.3 mg CE/dosage form, about 0.45 mg CE/dosage form, about 0.625 mg CE/dosage form, about 0.9 mg CE/dosage form, or about 1.25 mg CE/dosage form. Viewed alternatively, in such embodiments, the amount of CE/dosage form, based on the total weight of the solid dosage form (on a dry weight basis), is present from about 0.05 weight % to about 1.0 weight %, or further, from 0.1 weight % to about 0.3 weight %. The tablet core then can be coated with the coating composition described above, in an amount of from about 30 weight % to about 75 weight %, based on the total weight of the solid dosage form, to produce a coated tablet. An optional color coat and/or polish coat also may be applied, as described previously.

In certain embodiments, the solid dosage form comprises a core material and at least one coating, wherein the core material includes (based on the total weight of the solid dosage form on a dry weight basis) from about 0.1 weight % to about 0.3 weight % conjugated estrogens; from about 4 weight % to about 35 weight % of at least one sugar, for example, lactose monohydrate; from about 5 weight % to about 10 weight % of at least one binder, for example, microcrystalline cellulose; from about 10 weight % to about 35 weight % of at least one water soluble polymer, for example, hydroxypropyl methylcellulose; and from about 0 to about 1 weight % of at least one lubricant, for example, magnesium stearate. In some such embodiments, the coating includes at least one sugar, e.g., sucrose, in an amount from about 30 weight % to about 60 weight %, from about 35 weight % to about 55 weight %, or from about 35 weight % to about 45 weight %; at least one binder, e.g., microcrystalline cellulose, in an amount from about 5 weight % to about 10 weight %, from about 5.5 weight % to about 9 weight %, or from about 5.5 weight % to about 7.5 weight %; at least one hydroxyalkyl cellulose, e.g., hydroxypropyl cellulose, in an amount from about 3 weight % to about 10 weight %, from about 4 weight % to about 5.5 weight %; at least one water soluble polymer, e.g., hydroxypropyl methylcellulose, in an amount from about 15 weight % to about 50 weight %, from about 25 weight % to about 40 weight %, or from about 20 weight % to about 30 weight %; optionally, a therapeutic agent in an amount of up to about 3 weight %; optionally, a second polymer that is water soluble or water dispersible, for example such as polymethacrylate, in an amount of up to about 20 weight %, or in an amount from about 3 weight % to 20 weight %; and optionally, at least one plasticizer in an amount of up to about 8 weight %, from about 2 weight % to about 4 weight %, or from about 2.5 weight % to about 3.5 weight %. In some such embodiments, the solid dosage form further comprises a color coat, a polish coat, or both a color coat and a polish coat.

One of the primary advantages of the present invention is that a wide variety of tablet cores, prepared according to the various processes known in the art, can readily be coated with the coating composition of the present invention, utilizing the simple spray techniques more frequently associated with film-coating applications. Thus, in a further embodiment, the present invention is directed to processes that comprise providing a tablet core and applying, e.g., by spraying, onto the core a sugar coating composition, as previously described. The invention also is directed to the products of such process, e.g., a coated tablet core. It should be noted, however, that although utilizing such spray techniques provides certain advantages, it is by no means required that the composition be applied in this manner. Other methods for coating pharmaceutical dosage forms, such as, for example, use of a fluidized bed application process, are well known to those of ordinary skill in the art.

In some embodiments, the sugar coating compositions utilized in the processes of the invention include water and a solids component that comprises: from about 30 weight % to about 60 weight % of at least one sugar; from about 5 weight % to about 10 weight % of at least one binder; from about 3 weight % to about 10 weight % of at least one hydroxyalkyl cellulose; from about 15 weight % to about 50 weight % of at least one water soluble polymer; optionally, a therapeutic agent in an amount of up to about 3 weight %; optionally, a second polymer that is water soluble or water dispersible, in an amount of up to about 20 weight %; and optionally, at least one plasticizer in an amount of up to about 8 weight %. In some embodiments, the second polymer is present in an amount from about 3 weight % to about 20 weight %. In some embodiments, the solids component of the sugar coating composition comprises: from about 30 weight % to about 60 weight % sucrose; from about 5 weight % to about 10 weight % microcrystalline cellulose; from about 3 weight % to about 10 weight % hydroxypropyl cellulose; from about 15 weight % to about 50 weight % hydroxypropyl methylcellulose; optionally, up to about 3 weight % medroxyprogesterone acetate; optionally, up to about 20 weight % of a polymethacrylate; and optionally up to about 8 weight % polyethylene glycol. In further embodiments, the solids component of the sugar coating composition comprises: from about 35 weight % to about 55 weight % sucrose; from about 5.5 weight % to about 9 weight % microcrystalline cellulose; from about 4 weight % to about 7 weight % hydroxypropyl cellulose; from about 25 weight % to about 40 weight % hydroxypropyl methylcellulose; optionally, up to about 3 weight % medroxyprogesterone acetate; optionally, up to about 20 weight % of a polymethacrylate; and from about 2 weight % to about 4 weight % polyethylene glycol. In still further embodiments, the solids component of the sugar coating composition comprises: from about 35 weight % to about 45 weight % sucrose; from about 5.5 weight % to about 7.5 weight % microcrystalline cellulose; from about 4 weight % to about 5.5 weight % hydroxypropyl cellulose; from about 20 weight % to about 30 weight % hydroxypropyl methylcellulose; optionally, up to about 3 weight % medroxyprogesterone acetate; optionally, up to about 20 weight % of a polymethacrylate; and from about 2.5 weight % to about 3.5 weight % PEG 400. In some embodiments, the core material is as previously described.

In certain embodiments, the sugar coating composition is disposed directly onto the tablet core without a the need for intervening sealing layers, as are used typically in traditional sugar-coating methods. If desired, however, a sealing layer, such as shellac and other agents known to those in the art, may be applied to the tablet core prior to application of the sugar coating composition. In some embodiments, the sugar coating composition contains a therapeutic agent, such as medroxyprogesterone acetate, as previously described, and is disposed directly onto the tablet core without a non-therapeutic agent containing sugar coat being first applied, or an intervening sealing layer.

In some embodiments, the sugar coating composition is applied in a ratio of sugar coating to tablet core of from 3:1 to about 1:3, or in a ratio from about 2:1 to about 1:2, or in a ratio of from about 1.25:1 to about 1:1.25; and thus, providing a coated tablet. In some embodiments, the sugar coating composition is sprayed at an air flow rate of from about 500 cubic feet per minute to about 9000 cubic feet per minute; and further, at an air flow rate of from about 1000 cubic feet per minute to about 5000 cubic feet per minute. In some embodiments, the tablet core temperature is about 35° C. to about 50° C., while the inlet air temperature is about 50° C. to about 80° C. While the processes of the present invention further may include the steps of spraying a color coat and/or polish coat onto the sugar coat, such steps are optional, and all of the coating steps may be carried out in a single coating pan. Also, the step of printing a logo, trademark, word, symbol or the like optionally may be included in the processes of the present invention. Printing may be performed by any of the methods well known to those skilled in the art.

Thus, the process of the present invention may comprise the steps of providing tablet cores, placing the tablet cores into a coating pan, such as a perforated coating pan commonly utilized in film-coating applications such as a perforated pan with side-vents, then sequentially spraying the tablet cores with the sugar coating composition, the color coat, and the polish coat (if desired). Spray techniques for coating tablets are well known to those of skill in the art, and are described, for example, in Stuart C. Porter, “Coating of Pharmaceutical Dosage Forms,” Remington: The Science and Practice of Pharmacy, 20th Ed., Chap. 46, Alfonso R. Gennaro, ed., Philadelphia College of Pharmacy and Science, Philadelphia, Pa. (2000), herein incorporated by reference in its entirety.

The processes of the present invention are much simpler, less labor intensive, and less reliant upon operator expertise than the traditional sugar-coating techniques known in the prior art. Additionally, due to the unique combination of ingredients utilized in the sugar coating composition and the volume of sugar coating that is applied, the coated tablets produced by the processes of the present invention are remarkably hard, durable, and resistant to cracking, even when highly hygroscopic tablet cores are utilized. In some embodiments, a plurality of tablet cores coated with the sugar coating compositions of the present invention contain cracking in less than 6 percent of the coated tablet cores. In further such embodiments, the percentage of cracks is about 1 to about 5 percent; and in still further embodiments, less than 1 percent. Additionally, the coating provides an excellent barrier to prevent the release of odors from the tablet core, and to prevent atmospheric elements from reaching and degrading the therapeutic agent(s) in the tablet core. Thus, the coating compositions and processes described herein are particularly well suited for preparing solid dosage forms that utilize therapeutic agents or other materials having strong odors, such as, sulfur-containing compounds, in the core material.

Moreover, as discussed previously, the sugar coating composition itself can include one or more therapeutic agents, in addition to the therapeutic agent(s) that are present within the tablet core. Thus, therapeutic agents can be separated by their compartmentalization into either the core or the coating, to thereby minimize unwanted chemical interaction between the agents. Additionally, since the coating and tablet core can be designed to release their ingredients at different rates, the present invention may be utilized to provide, in a single dosage form, both a quick release and a sustained release formulation of the therapeutic agent(s). Also, it has been found unexpectedly that utilizing, in a sugar coating containing a therapeutic agent, a control release agent, which has been used traditionally for controlling drug diffusion from a therapeutic core, allows effective control of the therapeutic agent in the sugar coating itself. For example, in some embodiments, the second polymer of the sugar coating compositions of the invention functions as the aforementioned control release agent. In some embodiments, the second polymer, which is water soluble or water dispersible, includes one or more of a polyvinyl acetate, an ethylcellulose, a polymethacrylate or a polyvinyl alcohol, that will function to retard the release of the active therapeutic agent from the coating. In some preferred embodiments, the second polymer, which is water soluble or water dispersible, is a polymethacrylate, such as Eudragit® NE30D. The features of the coating compositions of the present invention, as described above, are particularly useful when delivering certain therapeutic agents such as hormones, where, for example, a quick release of one hormone, located in the coating, may be followed by a slow, sustained release of a second hormone from the tablet core. Of course, this property of the solid dosage forms also may be utilized to provide both a quick release and a sustained release of a single therapeutic agent, where both the tablet core and the coating contain the same therapeutic agent.

### EXAMPLES

The invention is further demonstrated in the following examples. The examples are for purposes of illustration and are not intended to limit the scope of the present invention.

### Example 1

### Preparation of 1.25 mg Conjugated Estrogen Coated Tablets

| | Amt/tablet |
|---|---|
| | (mg) |
| Tablet Core | |
| CE Desiccation with Lactose @ 42.9 mg/g | 29.14 |
| Lactose Monohydrate, NF (Spray Dried) | 120.3 |
| Microcrystalline Cellulose, NF | 36.0 |
| Hypromellose, USP, 2208, K100M (100,000 cps) | 54.0 |
| Magnesium Stearate, NF | 0.600 |
| Totals | 240.0 |
| Sugar Coat Filler Suspension (A) | |
| Hydroxypropyl Cellulose, NF | 13.80 |
| Hypromellose, USP, 2910, E5 (5 cps) | 59.8 |
| Hypromellose, USP, 2910, E15 (15 cps) | 15.00 |
| Microcrystalline Cellulose, NF | 18.40 |
| Polyethylene Glycol 400, NF | 8.05 |
| Sucrose, NF | 115.0 |
| Totals | 230 |
| Color Suspension (B) | |
| Opadry ® II, Yellow, 40L12916 | 15.00 |
| Polish Solution (C) | |
| Opaglos ® 2, Clear, 98Z19173 | 10.00 |
| Total Finished Tablet Weight | 495 |

### Tablet Core

1. Add the lactose monohydrate, NF, C.E. desiccation with lactose, microcrystalline cellulose, NF, and the Hypromellose, USP, 2208 (K100M Premium, CR) to a high shear mixer. Blend all ingredients with plows only.
2. Granulate the blend with water, U.S.P., purified, mixing with plows and choppers.
3. Size the granulation using a cutting mill.
4. Dry the granulation in a fluid bed dryer.
5. Size the dried granulation using a cutting mill.
6. Transfer the granulation to a V Blender. Blend.
7. Add the magnesium stearate, NF to the V Blender in step #6. Blend.
8. Compress granulation to target tablet weight, hardness and thickness using appropriately sized oval tooling on a rotary tablet press.

Similar formulations and manufacturing procedures have been developed and prepared for tablets containing CE at strengths of 0.3 mg, 0.45 mg, 0.625 mg and 0.9 mg.

### Sugar Coat Filler Suspension (A)

1. Place the purified water in a suitable stainless steel container.
2. With mixing, add the hydroxypropyl cellulose, hydroxypropyl methylcellulose, 2910, E5, hydroxypropyl methylcellulose, 2910, E15, and the polyethylene glycol 400 to Step #1.
3. With mixing, add the sucrose to Step #2 and mix until all ingredients are dissolved.
4. With mixing, add the microcrystalline cellulose to Step #3.
5. Allow Step #4 to de-aerate, bring to theoretical weight with purified water with mixing, and cool to room temperature prior to use, if necessary.
6. Continue mixing Step #5 until the filler application is complete.

### Color Suspension (B)

1. Add the purified water to a suitable stainless steel container.
2. With mixing, add the Opadry® II Yellow (Colorcon, West Point, Pa.) to Step #1. Continue stirring until the suspension is complete.
3. Bring Step #2 to theoretical weight with purified water, with mixing, if necessary.
4. Continue mixing Step #3 until the color application is complete.

### Polish Solution (C)

1. Add the purified water to a suitable stainless steel container.
2. With mixing, add the Opaglos® 2 Clear (Colorcon, West Point, Pa.) to Step #1. Continue mixing until the solution is complete.

Prior to application, allow the solution to de-aerate and bring Step #2 to theoretical weight with purified water with mixing, if necessary. Mixing is not necessary during the application.

### Tablet Coating

1. Load the compressed tablet cores in perforated coating pan(s).
2. Apply sufficient sugar coat filler suspension (A) to step #1 tablet cores to achieve desired coating weight gain above the average tablet core weight.
3. Apply sufficient color suspension (B) to step #2 filled tablets to achieve desired coating weight gain above the average filled tablet weight.
4. Apply sufficient polish solution (C) to step #3 colored tablets to achieve desired coating weight gain above the average colored tablet weight.

### Example 2

The tablet core composition utilized in Example 1 contains hydrogel (Hypromellose) type polymers, which are useful to modify/control the release of the active ingredient. This type of tablet core is flexible, and prone to swelling, however. A conventional sugar coat tends to be brittle and is prone to chipping, cracking and splitting due to processing conditions and/or if exposed to inappropriate mechanical stress (Pharmaceutical Coating Technology, Cole E, Hogan J., Aulton M., 1995, page 62, section 3.5). This example shows the ability of the coating composition of the present invention to resist cracking.

As controls, tablets containing hydrogel polymers and 1.25 mg/tablet of water-soluble estrogens, similar to the tablet cores described in Example 1, were coated with a conventional sugar coat. When exaggerated physical abuse was applied to the coated tablets, the vast majority of the tablets developed cracks in the coating. When the same tablets were coated with a sugar coat composition of the present invention, similar to the sugar coating suspension utilized in Example 1, and achieving the same coating weight gain, no cracks were observed in the coating. These data are presented in Table 1. Also included are data on traditional tablet cores (non-swelling) coated with a conventional sugar coat. These data also show that some coat cracking can be observed even on these more stable tablet cores with a conventional sugar coat.

TABLE 1
NEW TABLET FORMULATION
Coated Tablet Cracking with Exaggerated Physical Abuse

| | Number of | Total # of | Average |
|---|---|---|---|
| Formulation | Batches Tested | tablets tested | % Cracks |
| Hydrogel core and sugar | 7 | 7 × 100 = 700 | 0 |
| coating composition of the | | | |
| present invention | | | |
| Traditional core and | 5 | 5 × 100 = 500 | 14 |
| conventional Sugar Coat | | | |
| Hydrogel core and | 7 | 7 × 100 = 700 | 95 |
| conventional Sugar Coat | | | |

### Example 3

### Preparation of 0.45 mg CE/1.5 mg MPA Coated Tablets with Intervening Sugar Coat

In this example, 0.45 mg CE tablet cores were prepared and coated with a sugar coat suspension, in accordance with the formulation and manufacturing process of Example 1, except that the tablet core weight was 120 mg and the total solids filler sugar coat applied was 90 mg. An active filler suspension containing medroxyprogesterone acetate (MPA) then was applied followed by the color and polish coats, as described below. Alternatively, the active filler suspension could be sprayed directly onto the tablet cores without an intervening coating step (e.g., a first sugar coating of the present invention such as Example 4, below).

### 0.45 mg/Tablet CE/1.50 mg/Tablet MPA Preparation

| | Input/ | |
|---|---|---|
| | Dosage Unit | |
| Ingredient | Input | Unit |
| Sugar Coated Core | 210 | mg |
| Active MPA Filler Suspension Coat (D) | | |
| Medroxyprogesterone Acetate, USP | 1.5 | mg |
| Sucrose, NF | 50.5 | mg |
| Microcrystalline Cellulose, NF | 8.31 | mg |
| Hydroxypropyl Cellulose, NF | 6.23 | mg |
| Hypromellose, 2910, USP, E6 (6 cps) | 27.1 | mg |
| Hypromellose, 2910, USP, E15 (15 cps) | 6.75 | mg |
| Polyethylene Glycol 400, NF | 3.63 | mg |
| Purified Water, USP* | 474 | mg |
| Color Suspension (E) | | |
| Hypromellose, 2910, USP, E3 (3 cps) | 7.79 | mg |
| Polyethylene Glycol 400, NF | 0.780 | mg |
| Titanium Dioxide, USP | 2.86 | mg |
| Ferric Oxide, NF, Yellow | 0.571 | mg |
| Purified Water, USP* | 69.6 | mg |
| Polish Suspension (F) | | |
| Opaglos ® 2, Clear, 97W19196 | 6.0 | mg |
| Purified Water, USP* | 114 | mg |
| Total tablet weight | 332 | mg |

*Removed during processing.

The tablet core and sugar coat filler suspension are prepared substantially as set forth in Example 1.

### Active MPA Filler Suspension (D)

1. Place the purified water in a suitable stainless steel container.
2. With mixing, add the MPA powder until uniformly dispersed.
3. With mixing, add the Hypromellose, 2910, E6, Hypromellose, 2910, E15, hydroxypropyl cellulose, polyethylene glycol 400 and sucrose; mix until all ingredients are dissolved.
4. Allow suspension to de-aerate.
5. With mixing, add the microcrystalline cellulose.
6. Bring the suspension to theoretical weight with purified water with mixing, and continue mixing until the MPA filler application is complete.

### Color Suspension (E)

1. Place a portion of purified water to a suitable stainless steel container.
2. With mixing, add the ferric oxide and titanium dioxide; blend until uniform.
3. Add the balance of the purified water to a suitable stainless steel container.
4. With mixing, add the Hypromellose, E3 and PEG 400 and stir until dissolved.
5. Add the Step #2 color mixture and continue stirring until the suspension is complete.
6. Allow suspension to de-aerate and bring to theoretical weight with purified water. Continue mixing until the color application is complete.

### Polish Solution (F)

1. Add the purified water to a suitable stainless steel container.
2. With mixing, add the Opaglos® 2 Clear. Continue mixing until the solution is complete. Allow the solution to de-aerate and bring to theoretical weight with purified water with mixing, if necessary.

### Tablet Coating

1. Load the compressed tablet cores into perforated coating pan(s).
2. Apply sufficient sugar coat filler suspension (A) to Step #1 tablet cores to achieve an average filler solids weight of 90 mg (±5 mg) above the average compressed core weight.
3. Apply sufficient active filler suspension (D) to Step #2 tablet cores to achieve an average filler solids weight of 104 mg (±5 mg) above the average inert filled tablet core weight.
4. Apply sufficient color suspension (E) to Step #3 filled tablets to achieve an average color solids weight of 12 mg (±2 mg) above the average MPA filled tablet weight.
5. Apply sufficient polish solution (F) to Step #4 colored tablets to achieve an average total polish solids weight of 6 mg (±1 mg) above the average colored tablet weight.

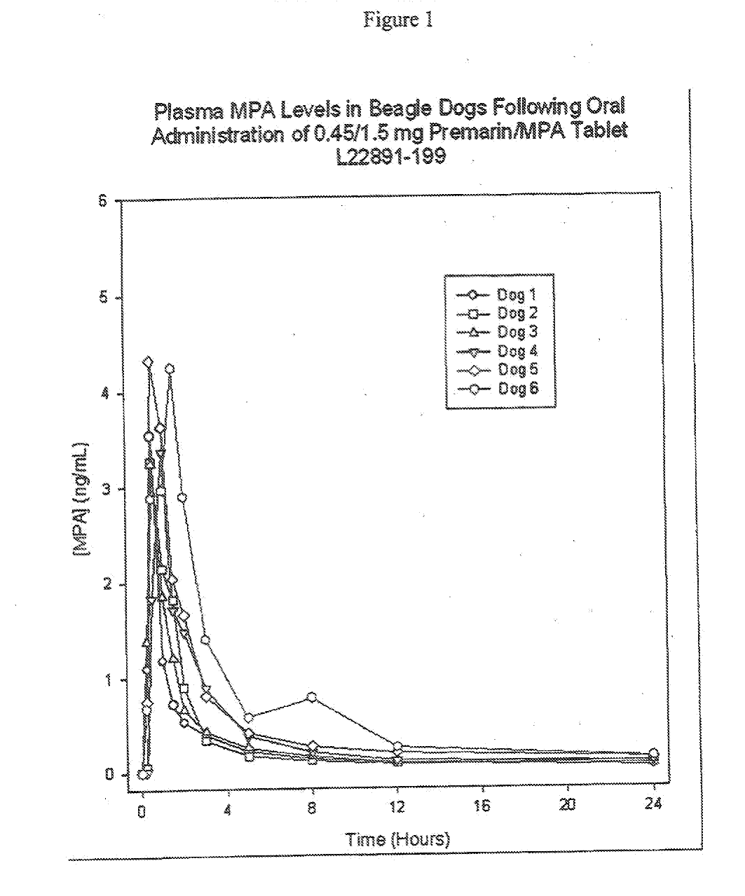

The utility of the 0.45 mg/1.5 mg CE/MPA tablet is further supported based on actual in vivo data from a pilot bioavailability study in dogs. FIG. 1 shows plasma MPA levels in 6 beagle dogs following oral administration of the subject tablet formulation. This data confirms that this type of formulation is capable of providing reproducible blood levels and is a viable delivery system for this class of drug.

### Example 4

### Preparation of 0.45 mg CE/1.5 mg MPA Coated Tablets—I MPA-Containing Coat Applied Directly

In this example, 0.45 mg CE tablet cores were prepared and coated with an active filler coat suspension, in accordance manufacturing process of Example 3, except the active filler coat suspension was sprayed directly onto the tablet core. That is to say, the coated tablet contained one sugar coat, which contained the MPA, and no intervening active-free sugar coat or sealing layer were applied initially to the tablet core.

### 0.45 mg/Tablet CE/1.50 mg/Tablet MPA Preparation

| | Input/ | |
|---|---|---|
| | Dosage Unit | |
| Ingredient | Input | Unit |
| Tablet Core | 120 | mg |
| CE Desiccation with Lactose @ 42.9 mg/g | 10.5 | mg |
| Lactose Monohydrate, NF (Spray Dried) | 58.2 | mg |
| Microcrystalline Cellulose, NF, EP | 18.0 | mg |
| Hypromellose, USP, 2208, K100M (100,000 cps) | 33.0 | mg |
| Magnesium Stearate, NF, EP | 0.300 | mg |
| Active MPA Filler Suspension Overcoat (G) | | |
| Medroxyprogesterone Acetate, USP | 1.5 | mg |
| Sucrose, NF | 36.5 | mg |
| Microcrystalline Cellulose, NF, EP | 6.09 | mg |
| Hydroxypropyl Cellulose, Klucel ® EF Pharma, NF | 4.57 | mg |
| Hypromellose, 2910, USP, E6 | 19.8 | mg |
| Hypromellose, 2910, USP, E15 | 4.95 | mg |
| Polyethylene Glycol 400, NF | 2.66 | mg |
| Polyacrylate dispersion 30% (Eudragit ® NE30D), EP | 46.5* | mg |
| Purified Water, USP** | 300 | mg |
| Color Suspension (H) | | |
| Hypromellose, 2910, USP, E6 | 3.40 | mg |
| Polyethylene Glycol 400, NF | 0.340 | mg |
| Titanium Dioxide, USP | 1.07 | mg |
| Ferric Oxide, NF, Yellow | 0.187 | mg |
| Purified Water, USP** | 30.7 | mg |
| Polish Suspension (I) | | |
| Opaglos ® 2, Clear, 98Z19173 | 2.0 | mg |
| Purified Water, USP** | 38 | mg |
| Total tablet weight | 217 | mg |

*13.9 mg solids
**Removed during processing.

The tablet core was prepared substantially as set forth in Example 1.

### Active MPA Filler Suspension (G)

1. Place the purified water in a suitable stainless steel container.
2. With mixing, add the MPA powder until uniformly dispersed.
3. With mixing, add the Hypromelloses, hydroxypropyl cellulose, polyethylene glycol 400 and sucrose; mix until all ingredients are uniformly dispersed.
4. With mixing, add the microcrystalline cellulose until uniformly dispersed. Cool the suspension to room temperature.
5. With mixing add the polyacrylate dispersion 30% (Eudragit® NE 30 D) EP through a stainless steel screen. Mix until uniformly dispersed.
6. Bring the suspension of Step #5 to theoretical weight with purified water with mixing, if necessary.
7. Continue mixing until the MPA filler application is complete.

### Color Suspension (H)

1. Add the purified water to a suitable stainless steel container.
2. With mixing, add the ferric oxide and titanium dioxide; blend until uniform.
3. Add the Hypromellose, 2910, USP, E6 and PEG 400; continue mixing until the suspension is complete.
4. Allow the suspension of Step #3 to de-aerate; and bring the suspension to theoretical weight with purified water with mixing, if necessary.
5. Continue mixing until the color application is complete.

### Polish Solution (I)

1. Add the purified water to a suitable stainless steel container.
2. With mixing, add the Opaglos® 2 Clear. Continue mixing until the solution is complete.
3. Prior to application, allow the solution to de-aerate and bring to theoretical weight with purified water with mixing, if necessary.

### Tablet Coating

1. Load the tablet cores into perforated coating pan(s).
2. Apply sufficient active filler suspension (G) to Step #1 tablet cores to achieve an average filler solids weight of 90 mg (±2 mg) above the average tablet core weight.
3. Cure the tablets at a tablet bed temperature of 45-50° C. for approximately 1 hour.
4. Apply sufficient color suspension (H) to Step #3 MPA filled tablets to achieve an average color solids weight of 5 mg (±1 mg) above the average MPA filled tablet weight.
5. Apply sufficient polish solution (I) to Step #4 colored tablets to achieve an average total polish solids weight of 2 mg (±1 mg) above the average colored tablet weight.

Similar formulations that have an active sugar fill coat containing MPA in an amount from about 0.5 to 10 mg can be prepared as described in any of the preceding Examples.

### Example 5

The following prophetic example describes the production of coated tablets of the present invention containing a blend of the synthetic conjugated estrogenic substances sodium estrone sulfate, sodium equilin sulfate, sodium 17α-dihydroequilin sulfate, sodium 17β-dihydroequilin sulfate, sodium 17α-estradiol sulfate, sodium 17β-estradiol sulfate, sodium equilenin sulfate, sodium 17α-dihydroequilenin sulfate, sodium 17β-dihydroequilenin sulfate. Utilizing procedures widely known to those of ordinary skill in the art, the conjugated estrogenic substances are mixed with commonly used excipients and compressed to form tablet cores of various strengths containing, for example, 0.3 mg, 0.45 mg, 0.625 mg, 0.9 mg, and 1.25 mg CE. The tablet cores then may be coated as described in any of the preceding examples.

### Example 6

### Dissolution Study of 0.45 mg/Tablet CE/1.50 mg/Tablet MPA Preparations

Since the active filler sugar coating and tablet core can be designed to release their ingredients at different rates, the present invention may be utilized to provide, in a single dosage form, both a quick release and a sustained release formulation of the therapeutic agent(s). The release time from the coating can be affected by, for example, the total solids of active applied to the core, the thickness of the active filler sugar coat applied, and the presence of a release controlling agent. This Example measured the MPA dissolution profiles of three formulations of 0.45 mg/tablet CE/1.50 mg/tablet MPA. The preparations were prepared as described in the preceding examples. All three preparations had the MPA filler sugar coat applied directly onto the CE tablet cores. The MPA filler sugar coat of one formulation further contained a second polymer, as described above, viz. polymethacrylate (15% Eudragit® NE30D), and the total solids of MPA sugar coat applied was 90 mg such as that of Example 4. The MPA sugar coat of the other two formulations were similar in composition to the Active Sugar Filler Suspension (D) and (S) of Example 3 and 11, respectively, i.e., with one water soluble polymer, with the total solids of MPA sugar coat applied being 90 mg and 200 mg, respectively. All three formulations were polished.

Percent MPA Dissolved^{#}

| Time | 1.5 mg MPA + 15% NE30D | 1.5 mg MPA | 1.5 mg MPA |
|---|---|---|---|
| (min.) | @ 90 mg | @ 200 mg | @ 90 mg |
| 15 | 2 | 10 | 12 |
| 30 | 4 | 27 | 42 |
| 45 | 7 | 44 | 67 |
| 60 | 10 | 60 | 83 |

^{#}Dissolution was performed at 50 rpm, in 0.54% SLS, 0.02M sodium acetate, pH 4.5 in standard vessels with sinkers;
n = 6.

The data confirms that a coating containing a second polymer, which traditionally has been used to delay release of therapeutic agents from a tablet core, can effectively delay the release of a therapeutic agent from the coating applied to a tablet core. The data also confirms that a greater amount of active filler sugar coat applied to the tablet core effectively can slow the release of the therapeutic agent compared to a lesser amount of active filler sugar coat similarly applied to the tablet core.

Examples 7-10 show the compositions of coated tablets of the invention containing 0.3 mg, 0.45 mg, 0.625 mg, 0.9 mg of conjugated estrogens, respectively. The tablet core and sugar coat filler suspension are prepared substantially as set forth in any of the preceding Examples.

### Example 7

### Composition of 0.3 Conjugated Estrogen Coated Tablet

| | Amt/tablet | |
|---|---|---|
| | (mg) | |
| Tablet Core | | |
| CE Desiccation with Lactose @ 4.29% CE | 6.993 | |
| Lactose Monohydrate (Spray Dried) | 61.7 | |
| Microcrystalline Cellulose | 18.0 | |
| Hypromellose 2208, K100M (100,000 cps) | 33.0 | |
| Magnesium Stearate | 0.300 | |
| Totals | 120 | mg |
| Sugar Coat Filler Suspension (J) | | |
| Sucrose | 45.0 | |
| Microcrystalline Cellulose | 7.20 | |
| Hydroxypropyl Cellulose | 5.40 | |
| Hypromellose, 2910, E6 (6 cps) | 23.4 | |
| Hypromellose, 2910, E15 (15 cps) | 5.85 | |
| Polyethylene Glycol 400 | 3.15 | |
| Totals | 90 | |
| Color Suspension (K) | | |
| Opadry ® Green | 5.00 | |
| Polish Solution (L) | | |
| Opaglos ® 2, Clear | 3.00 | |
| Total Finished Tablet Weight | 218 | |

### Example 8

### Composition of 0.45 mg Conjugated Estrogen Coated Tablets

| | Amt/tablet | |
|---|---|---|
| | (mg) | |
| Tablet Core | | |
| CE Desiccation with Lactose @ 4.29% CE | 10.4895 | |
| Lactose Monohydrate (Spray Dried) | 58.2 | |
| Microcrystalline Cellulose | 18.0 | |
| Hypromellose 2208, K100M (100,000 cps) | 33.0 | |
| Magnesium Stearate | 0.300 | |
| Totals | 120 | mg |
| Sugar Coat Filler Suspension (M) | | |
| Sucrose | 45.0 | |
| Microcrystalline Cellulose | 7.20 | |
| Hydroxypropyl Cellulose | 5.40 | |
| Hypromellose, 2910, E6 (6 cps) | 23.4 | |
| Hypromellose, 2910, E15 (15 cps) | 5.85 | |
| Polyethylene Glycol 400 | 3.15 | |
| Totals | 90 | |
| Color Suspension (N) | | |
| Opadry ® Blue | 5.00 | |
| Polish Solution (O) | | |
| Opaglos ® 2, Clear | 3.00 | |
| Total Finished Tablet Weight | 218 | |

### Example 9

### Composition of 0.625 mg Conjugated Estrogen Coated Tablets

| | Amt/tablet | |
|---|---|---|
| | (mg) | |
| Tablet Core | | |
| CE Desiccation with Lactose @ 4.29% CE | 14.5688 | |
| Lactose Monohydrate (Spray Dried) | 54.1 | |
| Microcrystalline Cellulose | 18.0 | |
| Hypromellose 2208, K100M (100,000 cps) | 33.0 | |
| Magnesium Stearate | 0.300 | |
| Totals | 120 | mg |
| Sugar Coat Filler Suspension (P) | | |
| Sucrose | 45.0 | |
| Microcrystalline Cellulose | 7.20 | |
| Hydroxypropyl Cellulose | 5.40 | |
| Hypromellose, 2910, E6 (6 cps) | 23.4 | |
| Hypromellose, 2910, E15 (15 cps) | 5.85 | |
| Polyethylene Glycol 400 | 3.15 | |
| Totals | 90 | |
| Color Suspension (Q) | | |
| Opadry ® Maroon | 5.00 | |
| Polish Solution (R) | | |
| Opaglos ® 2, Clear | 3.00 | |
| Total Finished Tablet Weight | 218 | |

### Example 10

### Composition of 0.9 mg Conjugated Estrogen Coated Tablets

| | Amt/tablet | |
|---|---|---|
| | (mg) | |
| Tablet Core | | |
| CE Desiccation with Lactose @ 4.29% CE | 20.979 | |
| Lactose Monohydrate (Spray Dried) | 78.0 | |
| Microcrystalline Cellulose | 26.0 | |
| Hypromellose 2208, K100M (100,000 cps) | 47.6 | |
| Magnesium Stearate | 0.433 | |
| Totals | 173 | mg |
| Sugar Coat Filler Suspension (S) | | |
| Sucrose | 65.0 | |
| Microcrystalline Cellulose | 10.4 | |
| Hydroxypropyl Cellulose | 7.80 | |
| Hypromellose, 2910, E6 (6 cps) | 33.8 | |
| Hypromellose, 2910, E15 (15 cps) | 8.45 | |
| Polyethylene Glycol 400 | 4.55 | |
| Totals | 130 | |
| Color Suspension (T) | | |
| Opadry ® White | 7.00 | |
| Polish Solution (U) | | |
| Opaglos ® 2, Clear | 3.00 | |
| Total Finished Tablet Weight | 313 | |

Example 11 shows the composition of a coated tablet of the invention containing 0.45 mg of conjugated estrogens, and 1.50 mg of MPA.

### Example 11

### Composition of 0.45 mg CE/1.5 mg MPA Coated Tablets—II MPA-Containing Coat Applied Directly

| | Amt/tablet | |
|---|---|---|
| | (mg) | |
| Tablet Core | | |
| CE Desiccation with Lactose @ 4.29% mg/g CE | 10.49 | |
| Lactose Monohydrate, NF/EP (powder) | 58.21 | |
| Microcrystalline Cellulose, NF/EP | 18.0 | |
| Hypromellose, USP, 2208 (100,000 cps) (K100M | 33.0 | |
| Prem, CR) | | |
| Magnesium Stearate, NF/EP, Hyqual Vegetable Code | 0.300 | |
| Totals | 120 | mg |
| Active MPA Filler Suspension Overcoat (S) | | |
| Medroxyprogesterone Acetate, USP/EP | 1.500 | |
| Sucrose, NF | 98.50 | |
| Microcrystalline Cellulose, NF/EP | 16.00 | |
| Hydroxypropyl Cellulose, Klucel EF Pharma, NF | 12.00 | |
| Hypromellose, 2910, USP/EP E6 | 52.00 | |
| Hypromellose, 2910, USP E15 | 13.00 | |
| Polyethylene Glycol 400, NF | 7.000 | |
| Totals | 200 | |
| Color Suspension (T) | | |
| Spectrablend Yellow | 6.00 | |
| Polish Solution (U) | | |
| Opaglos ® 2, Clear | 1.50 | |
| Total Finished Tablet Weight | 328 | |

The disclosures of each patent, patent application and publication, including books, cited or described in this document are incorporated herein by reference in their entirety.

Various modifications of the invention, in addition to those described herein, will be apparent to those skilled in the art from the foregoing description. Such modifications also are intended to fall within the scope of the appended claims.

What is claimed is:

1. A composition comprising water and a solids component that comprises:
from about 30 weight % to about 60 weight % of at least one sugar;
from about 5 weight % to about 10 weight % of at least one binder;
from about 3 weight % to about 10 weight % of at least one hydroxyalkyl cellulose;
from about 15 weight % to about 50 weight % of at least one water soluble polymer;
optionally, a therapeutic agent in an amount of up to about 3 weight %;
optionally, a second polymer that is water soluble or water dispersible, in an amount of up to about 20 weight %; and
optionally, at least one plasticizer in an amount of up to about 8 weight %.

2. The composition of claim 1 that is in the form of an aqueous suspension.

3. The composition of claim 1 that comprises:
from about 75 weight % to about 85 weight % water; and
from about 15 weight % to about 25 weight % of the solids component.

4. The composition of claim 1, wherein the binder comprises microcrystalline cellulose.

5. The composition of claim 4, wherein the hydroxyalkyl cellulose comprises hydroxyethyl cellulose or hydroxypropyl cellulose.

6. The composition of claim 5, wherein the hydroxyalkyl cellulose comprises hydroxypropyl cellulose.

7. The composition of claim 1, wherein the water soluble polymer comprises hydroxypropyl methylcelluose, acacia, sodium carboxymethylcellulose, dextrin, alginic acid, gelatin, guar gum, methylcellulose, sodium alginate, zein, polyvinylpyrrolidone, vinylpyrrolidine-vinyl acetate copolymer, vinyl acetate-crotonic acid copolymer, or ethyl acrylate-methacrylate acid copolymer.

8. The composition of claim 7, wherein the water soluble polymer comprises hydroxypropyl methylcelluose, sodium carboxymethylcellulose, or methylcellulose.

9. The composition of claim 8, wherein the water soluble polymer comprises hydroxypropyl methylcellulose.

10. The composition of claim 1, wherein the plasticizer comprises propylene glycol, glycerin, trimethylolpropane, polyethylene glycol polymers, dibutyl sebacate, acetylated monoglycerides, diethylphthalate, triacetin, glyceryltriacetate, acetyltrietyhyl citrate, or triethyl citrate.

11. The composition of claim 10, wherein the plasticizer comprises polyethylene glycol.

12. The composition of claim 1, wherein the sugar comprises sucrose, dextrose, maltose, glucose, fructose, galactose, mannose, lactose, trehalose, lactulose, levulose, raffinose, ribose, xylose, sorbitol, mannitol, xylitol, erythritol, maltitol, lactitol, isomalt, or polyalditol.

13. The composition of claim 12, wherein the sugar comprises sucrose.

14. The composition of claim 1, wherein:
the sugar comprises sucrose;
the binder comprises microcrystalline cellulose;
the hydroxyalkyl cellulose comprises hydroxypropyl cellulose;
the water soluble polymer comprises hydroxypropyl methylcellulose;
the therapeutic agent, when present, comprises medroxyprogesterone acetate;
the second polymer, when present, comprises a polyvinyl acetate, an ethylcellulose, a polymethacrylate, or a polyvinyl alcohol; and
the plasticizer, when present, comprises polyethylene glycol.

15. The composition of claim 1, wherein the solids component comprises:
from about 35 weight % to about 55 weight % of at least one sugar;
from about 5.5 weight % to about 9 weight % of at least one binder;
from about 4 weight % to about 7 weight % of at least one hydroxyalkyl cellulose;
from about 25 weight % to about 40 weight % of at least one water soluble polymer;
optionally, from about 0.001 weight % to about 3 weight % of a therapeutic agent;
optionally, from about 3 weight % to about 20 weight % of a second polymer that is water soluble or water dispersible; and
from about 2 weight % to about 4 weight % at least one plasticizer.

16. The composition of claim 15, wherein:
the sugar comprises sucrose;
the binder comprises microcrystalline cellulose;
the hydroxyalkyl cellulose comprises hydroxypropyl cellulose;
the water soluble polymer comprises hydroxypropyl methylcellulose;
the therapeutic agent, when present, comprises medroxyprogesterone acetate;
the second polymer, when present, comprises a polyvinyl acetate, an ethylcellulose, a polymethacrylate, or a polyvinyl alcohol; and
the plasticizer comprises polyethylene glycol.

17. The composition of claim 1, wherein the solids component comprises:
from about 35 weight % to about 45 weight % of at least one sugar;
from about 5.5 weight % to about 7.5 weight % of at least one binder;
from about 4 weight % to about 5.5 weight % of at least one hydroxyalkyl cellulose;
from about 20 weight % to about 30 weight % of at least one water soluble polymer;
optionally, from about 0.001 weight % to about 3 weight % of a therapeutic agent;
optionally, from about 3 weight % to about 20 weight % of a second polymer that is water soluble or water dispersible; and
from about 2.5 weight % to about 3.5 weight % of at least one plasticizer.

18. The composition of claim 17, wherein:
the sugar comprises sucrose;
the binder comprises microcrystalline cellulose;
the hydroxyalkyl cellulose comprises hydroxypropyl cellulose;
the water soluble polymer comprises hydroxypropyl methylcellulose;
the therapeutic agent, when present, comprises medroxyprogesterone acetate;
the second polymer, when present, comprises a polyvinyl acetate, an ethylcellulose, a polymethacrylate, or a polyvinyl alcohol; and
the plasticizer comprises polyethylene glycol.

19. The composition of claim 1, wherein the composition comprises a therapeutic agent.

20. The composition of claim 19, wherein the therapeutic agent is medroxyprogesterone acetate.

21. The composition of claim 20, wherein the medroxyprogesterone acetate is in an amount from about 0.5 mg to about 10 mg.

22. The composition of claim 1, wherein the composition comprises a second polymer, which is water soluble or water dispersible.
